(19) United States
(12) Patent Application Publication
(10) Pub. No.: US 2007/0154570 A1
(43) Pub. Date: Jul. 5, 2007

(54) Use of nitric oxide in the treatment and disinfection of biofilms

(76) Inventors: Christopher C. Miller, North Vancouver (CA); Douglas R. Hole, Edmonton (CA)

Correspondence Address: Carissa A. Tener;SIDLEY AUSTIN LLP, Suite 4000, 555 West Fifth Street, Los Angeles, CA, 90013, US

(21) Appl. No.: 11/592,950

(22) Filed: Nov. 1, 2006

Related U.S. Application Data
(63) Continuation-in-part of application No. 10/953,827, filed on Sep. 29, 2004.

(51) Int. Cl. A01N 59/00; A61K 33/00

(52) U.S. Cl. 424/718

(57) ABSTRACT

The administration of gaseous nitric oxide as a biocidal moiety is proffered as a de novo treatment in the control and eradication of biofilms. The present invention relates to the use or methods of application of exogenous nitric oxide gas (gNO) as a stand alone biocidal agent or in cohort with any or all adjunct vehicles in the control of biofilms generated by microbial organisms, i.e., bacteria, protozoa, amoeba, fungi etc. Further, the present invention introduces the concept of utilization and methods of application of gaseous nitric oxide in control and eradication of biofilm forming microorganisms. Other embodiments include the use of a nitric oxide releasing material to eradicate and-control the growth of biofilms. Another embodiment includes the use of a gaseous nitric oxide releasing material packaged in an air-tight container with a medical device to prevent the growth of biofilm on the medical device.

### CLAIM OF PRIORITY

This application is a continuation-in-part application of and claims priority to U.S. patent application Ser. No. 10/953,827 filed on Sep. 29, 2004, which claims priority to U.S. Provisional Patent Application Ser. No. 60/506,807 filed on Sep. 29, 2003, each of which are herein incorporated by reference in its entirety.

### FIELD 0F THE INVENTION

The present invention generally relates to a methodology for obtunding biofilms.

### BACKGROUND OF INVENTION

Microbial organisms are capable of adhering to a surface aggregate in a polymer-like matrix. This is referred to as a biofilm and is synthesized endogenously by the microbe(s). Biofilms are ubiquitous in nature and are commonly found in a wide range of environments including domestic and industrial water systems. Biofilms are also etiologic agents for a number of disease states in mammals. Otitis media, dental plaque, bacterial endocarditis, cystic fibrosis and Legionnair's disease along with a broad array of hospital acquired, dental and medical clinic infections are examples of its pathology. Bacteria growing in biofilms display increased resistance to antibiotics. Commonly surveyed microbial organisms that form biofilms are Burkholderia cenocepacia, Staphlococcus, Steptococccus, Pseudomonas, and Legionnella and their subtypes.

In U.S. Pat. No. 5,957,880, Igo taught that adding nitric oxide to blood within an extracorporeal system is known to inhibit platelet activation. Our summary of Igo's '880 reference is based on Igo's teaching which is as follows (bracketed material is added and underlining was added for emphasis):

Referring to FIG. 1, a typical CPB circuit is indicated generally by reference numeral 10. The patient is shown by numeral 12. A venous cannula 13 inserted into the patient is connected into a fluid inlet tube 14 that directs blood from the patient to a venous reservoir 18. Another cannula 15 inserted in the patient is connected to another fluid inlet 16 that also leads from the patient to venous reservoir 18. Reservoir 18 may be a pole mounted unit or may be located on the heart-lung machine table, but in either case normally is the first fixed point in the circuit, lines 14 and 16 normally being flexible and long enough o allow surgeon and surgical assistants room to maneuver around the surgical table. The purpose of venous reservoir 18 is to accumulate the admitted blood for feeding the balance of the CPB circuit. The accumulator eliminates pump starvation and cessation of pump prime by providing a buffer from ebb and flow of blood from the patient.

From the venous reservoir, plastic tubing 20 leads to the inlet side of a roller pump 22. Roller pump 22 has a hub 24 from which protrude two arms 26. These arms impinge on the tubing 20 collapsing it. Rotation of the pump hub 24 in the direction indicated by reference numeral 28 provides the desired flow direction and flow rate. The blood leaves the roller pump 22 through tubing 30 to the inlet of the oxygenator 32. The blood can be thermally adjusted by passing it from the oxygenator 32 through tubing 34 into a heat exchanger 36 for heating and cooling before returning to the oxygenator 32 by tubing 38. Upon oxygenation, the blood exits the oxygenator in two ways. The first way is through tubing 40 to another roller pump 42, from there pumped through tubing 44 to a cardioplegia system 46, then to the patient 12 though outlet tubing 47 and a cannula 48. The other mechanism with which the blood leaves the oxygenator 32 is through tubing 50. A filter 52 is located on a side branch of this portion of the circuit. When it is desired to use the filter 52, tubing 50 is clamped in the area noted by numeral 54 and the blood travels through the filter 52 before returning to the patient through outlet tubing 57 and a cannula 56. The venous return reservoir 18 is the juncture of all blood removed from the patient. It is at this location where the improvement according to this invention suitably may be added to the CPB circuit, prior to the pump 22 and the blood treatment oxygenator 32.

FIG. 2 depicts an extracorporeal blood treatment circuit in general, designated by reference numeral 11, and in which reference numerals are the same for the like elements found in the specific CPB circuit shown in FIG. 1. Reference numeral 41 represents a blood treatment component. In the case of a CPB apparatus as in FIG. 1, blood treatment component 41 comprises at least oxygenator 32 and optionally also heat exchanger 36 with connecting tubing 34, 38 and either or both (1) the cardioplegia system 46 with associated second pump 42 and connecting tubing 40, 44, 47 and (2) the filter 52 with associated tubing 50. Numeral 17 indicates a blood fluid inlet generally and numeral 49 indicates a fluid outlet for blood return generally to the patient in FIG. 2. In accordance with this invention, blood treatment component 41 of the fluid circuit of the apparatus 11, instead of being an oxygenation system as in FIG. 1, suitably may be a heat exchange system 36, a renal dialysis component for exchange of urea and other blood chemicals with a dialysate solution across an exchange membrane, or an organ perfusion component such as an ex vivo liver and perfusion support system tying into circuit interconnects 30 and 49.

In accordance with this invention, one or more feeds of nitric oxide are employed, as necessary in the particular circuit, to maintain the concentration of nitric oxide in the circulating extracorporeal blood at a dosage effective to produce the desired inhibition of platelet activation over a period of time sufficient for the journey through the extracorporeal circulation apparatus yet insufficient to sustain the inhibition after the blood is returned to the patient and desired dosages. FIG. 3 depicts one such feed at the initial (venous inlet) portion of the circuit illustrated in FIG. 1. In this preferred embodiment of the invention, a gas permeable membrane 60 is located within a conduit 62 of the blood circuit located immediately downstream from the reservoir 18. The gas permeable membrane 60 is elongated and tubular inform and is disposed longitudinally within conduit 62 adapted to come into contact with blood flowing through conduit 62. A gaseous source, a mixture of nitric oxide and a carrier gas such as nitrogen, is housed in container 68 under high pressure. Regulator 66 controls the output gas pressure to periodic driver 69. The purpose of the periodic driver 69 is to induce a sinusoidal shaped pressure curve to the gas much like a “pulse.” The gas leaves the driver through tubing 64 and flows into the interior of gas permeable membrane 60. Due to the permeability of this membrane 60 to nitric oxide gas, the gas will diffuse through the membrane and dissolve in the blood plasma where it will come into contact with platelets. The membrane is selected to be impermeable to nitrogen and the nitrogen carrier gas will not diffuse through the membrane. Coupled to the outlet of the membrane 60 is outlet tubing 61, which is connected to valve 63. Valve 63 adjusts the back pressure of the system. From the valve 63 the carrier gas and any residual nitric oxide gas is carried through tube 65 into container 67, which is tilled with a scavenger liquid such as methylene blue. The gas mixture is allowed to bubble up through the container containing the scavenger liquid. The scavenger liquid absorbs any residual nitric oxide so that the only gas that escapes into the atmosphere is the carrier gas.

Blood guarded by dissolved nitric oxide exits conduit 62 and into tubing 20 where it passes by a conventional blood flow measuring device 90. Signals from blood flow measuring device 90 are transferred by line 92 to controller feedback logic component 94 which outputs a signal through line 96 to controller driver component 98 for controlling pressure and flow from regulator 66. The controller system comprising units 90, 94 and 98 with connecting lines 92 and 96 controls the flow of gas into membrane 60 in relation to the flow of blood through tubing 20. In this manner, when the flow rate of the blood is low, the nitric oxide introduction is correspondingly and automatically reduced. Conversely, in cases of high flow the nitric oxide introduction is correspondingly and automatically raised.

The gas permeable membrane 62 has a gas permeable rate K which is dependent on the material of construction and the molecular characteristics of the gas. For nitric oxide, the gaseous release rate from membrane 60 is proportional to K, the exposed surface of the membrane to the blood, the internal gaseous pressure within the membrane and the hydraulic pressure of and gas tension of nitric oxide (if any) in the blood flowing by it. Delivered molecular concentrations to the blood is [sic] calculated knowing the above plus the absorption coefficient of the blood to the nitric oxide. Thus the controller controls the gas flow and at a level which, for the characteristics of membrane 60 and the absorption coefficient of nitric oxide gas at the temperature of the blood in the apparatus (before thermal adjustment, if any), is sufficient to provide an actual concentration of nitric oxide in solution effective in the presence of venous red blood cell blood hemoglobin to inhibit platelet activation.

FIG. 4 illustrates a longitudinal sectional view of the conduit 62, the gas permeable membrane 60 and the tubing 64. Nitric oxide gas flows into the membrane 60 at location 70. As the gas pressure inside the gas permeable membrane 60 exceeds the pressure of the blood within conduit 62, nitric oxide gas will diffuse from the membrane into the blood stream as indicated by arrows 74. The nitric oxide will be absorbed by the blood cellular components which will mediate the inflammatory response as described earlier.

Referring to FIG. 5, which illustrates a cross section of FIG. 3 along the line A-A, the relationship between the geometry's of the conduit 62 and gas permeable membrane 60 is as follows. The cross sectional area of the inside of conduit 62 minus the sectional area of the gas permeable membrane 60 (such difference being referenced by numeral 76) is approximately equivalent to the cross section of the tubing elsewhere in the CPB circuit, (i.e. the cross section of tubing element 20). With this relationship the blood is not subjected to an adverse pressure gradient in conduit 62. Longitudinally, the shape of the gas permeable membrane 60 follows that of the conduit 62, again so that adverse pressure gradients are not imparted into the circuit.

FIG. 6 illustrates another preferred embodiment of the invention. In this embodiment a carrier gas is not used so that container 68 holds a 100% concentration of nitric oxide. A pulse drive generator 69 is not shown but may be present. In this embodiment, there is no outlet conduit of membrane 60. As pressure builds up in conduit 60, the nitric oxide diffuses into the bloodstream as previously described. Because there are no residual carrier gas molecules, there is no need for a return. Simply stated, components 61, 63, 65, and 67 of the embodiment depicted in FIG. 2 are absent at the distal end of membrane 60 and the tube 62 in this configuration. As in the embodiment depicted in FIG. 3, a controller comprising components 90, 94 and 98 with connections 92 and 96 controls the concentration of nitric oxide in solution in the blood. FIG. 8 illustrates a cross sectional view B-B of FIG. 7 with the same numbers used in the same way as in FIG. 5.

The above embodiments illustrate an optimal configuration of the invention in which the blood flows around the external portion of a gas permeable membrane 60. While it is within the scope of this invention that the system can be configured so that the gas is on the external portion of the membrane and blood is flowed within the membrane, in low gas pressure conditions some membranes dilate, increasing the cross sectional area of the membrane and lowering blood flow through that portion of the apparatus, and in high gas pressure conditions, some membranes might collapse, reducing blood flow. In the preferred embodiments, if gas flow is zero, the membrane might collapse but it would not occlude or preclude blood flow.

FIG. 9 depicts another embodiment of the [Igo] invention. In this embodiment the nitric oxide feed is to reservoir 18. The feed comprises a diffuser 100 for diffusing nitric oxide gas into the reservoir, and comprises a regulator 66 for controlling gas pressure and rate of flow into the reservoir and a driver 69 for delivering the nitric oxide gas into reservoir 18 through inlet 64 in a pulsatile manner. Suitably diffuser 100 comprises a membrane or filter 80 that is not permeable to blood and is permeable to nitric oxide gas through which nitric oxide gas is introduced into the reservoir. As in the embodiment depicted in FIGS. 3 and 6, a controller comprising components 90, 94 and 98 with connections 92 and 96 controls the concentration of nitric oxide in solution in the blood.

It is important that the location of the nitric oxide feed be close to the patient cannulation point as possible in the extracorporeal circuit to reduce so much as practicable the period of exposure of platelets to non-endothelial surfaces. At least one feed location is described generally as upstream of the pump that is needed to circulate the blood extracorporeally through the system and back to the patient. With reference to the FIG. 2, that point is anywhere in line 15. In FIGS. 3-9, which involve a CPB circuit where blood from two inlets 14 and 16 is pooled in reservoir 18, either the reservoir or the tubing immediately past the reservoir is selected for initial introduction of the nitric oxide, for the practical reason that these are the closest stationary locations in the system to the patient source of blood and also because control of nitric oxide introduction is most readily accomplished in the reservoir or in the blood filled lines in the immediately downstream tubing under the influence of a pump as opposed to in the blood inlet lines where lines are mobile to allow access to the surgical field, and especially in the case of blood suctioned from the operative field where intermittent blood and air flow occurs. The closest stationary location will vary according to the blood treatment component 41 involved in the use of this invention. Because of the very short half life of nitric oxide in the blood, additional feeds may be used further downstream to maintain the desired nitric oxide concentration in the blood without overdosing the blood in but one location.

In other words, Igo teaches away from adding nitric oxide to blood to combat pathogens.

In U.S. Pat. No. 6,432,077, Stenzler teaches that topical application of nitric oxide to wounds and/or skin of mammals is beneficial to wound healing because it decreases further infection. No where does Stenzler teach, disclose or suggest exposing nitric oxide to blood to combat pathogens. Our summary of Stenzler is based on his disclosure, which reads as follows:

The treatment of infected surface or subsurface lesions in patients has typically involved the topical or systemic administration of anti-infective agents to a patient. Antibiotics are one such class of anti-infective agents that are commonly used to treat an infected abscess, lesion, wound, or the like. Unfortunately, an increasingly number of infective agents such as bacteria have become resistant to conventional antibiotic therapy. Indeed, the increased use of antibiotics by the medical community has led to a commensurate increase in resistant strains of bacteria that do not respond to traditional or even newly developed anti-bacterial agents. Even when new anti-infective agents are developed, these agents are extremely expensive and available only to a limited patient population.

Another problem with conventional anti-infective agents is that some patients are allergic to the very compounds necessary to treat their infection. For these patients, only few drugs might be available to treat the infection. If the patient is infected with a strain of bacteria that does not respond well to substitute therapies, the patient's life can be in danger.

A separate problem related to conventional treatment of surface or subsurface infections is that the infective agent interferes with the circulation of blood within the infected region. It is sometimes the case that the infective agent causes constriction of the capillaries or other small blood vessels in the infected region which reduces bloodflow. When bloodflow is reduced, a lower level of anti-infective agent can be delivered to the infected region. In addition, the infection can take a much longer time to heal when bloodflow is restricted to the infected area. This increases the total amount of drug that must be administered to the patient, thereby increasing the cost of using such drugs. Topical agents may sometimes be applied over the infected region. However, topical anti-infective agents do not penetrate deep within the skin where a significant portion of the bacteria often reside. Topical treatments of anti-infective agents are often less effective at eliminating infection than systemic administration (i.e., oral administration) of an anti-infective pharmaceutical.

In the 1980's, it was discovered by researchers that the endothelium tissue of the human body produced nitric oxide (NO), and that NO is an endogenous vasodilator, namely, and agent that widens the internal diameter of blood vessels. NO is most commonly known as an environmental pollutant that is produced as a byproduct of combustion. At high concentrations, NO is toxic to humans. At low concentrations, researchers have discovered that inhaled NO can be used to treat various pulmonary diseases in patients. For example, NO has been investigated for the treatment of patients with increased airway resistance as a result of emphysema, chronic bronchitis, asthma, adult respiratory distress syndrome (ARDS), and chronic obstructive pulmonary disease (COPD).

NO has also been investigated for its use as a sterilizing agent. It has been discovered that NO will interfere with or kill the growth of bacteria grown in vitro. PCT International Application No. PCT/CA99/01123 published Jun. 2, 2000 discloses a method and apparatus for the treatment of respiratory infections by NO inhalation. NO has been found to have either an inhibitory and/or a cidal effect on pathogenic cells.

While NO has shown promise with respect to certain medical applications, delivery methods and devices must cope with certain problems inherent with gaseous NO delivery. First, exposure to high concentrations of NO is toxic, especially exposure to NO in concentrations over 1000 ppm. Even lower levels of NO, however, can be harmful if the time of exposure is relatively high. For example, the Occupational Safety and Health Administration (OSHA) has set exposure limits for NO in the workplace at 25 ppm time-weighted averaged for eight (8) hours. It is extremely important that any device or system for delivering No include features that prevent the leaking of NO into the surrounding environment. If the device is used within a closed space, such as a hospital room or at home, dangerously high levels of NO can build up in a short period of time.

Another problem with the delivery of NO is that NO rapidly oxidizes in the presence of oxygen to form NO₂, which is highly toxic, even at low levels. If the delivery device contains a leak, unacceptably high levels NO₂ of can develop. In addition, to the extent that NO oxides to form NO₂, there is less NO available for the desired therapeutic effect. The rate of oxidation of NO to NO₂ is dependent on numerous factors, including the concentration of NO, the concentration of O₂, and the time available for reaction. Since NO will react with the oxygen in the air to convert to NO₂, it is desirable to have minimal contact between the NO gas and the outside environment.

Accordingly, there is a need for a device and method for the treatment of surface and subsurface infections by the topical application of NO. The device is preferably leak proof to the largest extent possible to avoid a dangerous build up of NO and NO₂ concentrations. In addition, the device should deliver NO to the infected region of the patient without allowing the introduction of air that would otherwise react with NO to produce NO₂. The application of NO to the infected region preferably decreases the time required to heal the infected area by reducing pathogen levels. The device preferably includes a NO and NO₂ absorber or scrubber that will remove or chemically alter NO and NO₂ prior to discharge of the air from the delivery device.

In a first aspect of the [Stenzler] invention, a device for the topical delivery of nitric oxide gas to an infected area of skin includes a source of nitric oxide gas, a bathing unit, a flow control valve. and vacuum unit. The bathing unit is in fluid communication with the source of nitric oxide gas and is adapted for surrounding the area of infected skin and forming a substantially air-tight seal with the skin surface. The flow control valve is positioned downstream of the source of nitric oxide and upstream of the bathing unit for controlling the amount of nitric oxide gas that is delivered to the bathing unit. The vacuum unit is positioned downstream of the bathing unit for withdrawing gas from the bathing unit.

In a second aspect of the [Stenzler) invention, the device according to the first aspect of the invention includes a controller for controlling the operation of the flow control valve and the vacuum unit.

In a third aspect of the [Stenzler] invention, the device according to the first aspect of the invention further includes a source of dilutent gas and a gas blender. The dilutent gas and the nitric oxide gas are mixed by the gas blender. The device also includes a nitric oxide gas absorber unit that is positioned upstream of the vacuum unit. The device also includes a controller for controlling the operation of the flow control valve and the vacuum unit.

In a fourth aspect of the [Stenzler] invention, a method of delivering an effective amount of nitric oxide to an infected area of skin includes the steps of providing a bathing unit around the infected area of skin, the bathing unit forming a substantially air-tight seal with the skin. Gas containing nitric oxide is then transported to the bathing unit so as to bathe the infected area of skin with gaseous nitric oxide. Finally, at least a portion of the nitric oxide gas is evacuated from the bathing unit.

It is an object of the [Stenzler] invention to provide a delivery device for the topical delivery of a NO-containing gas to an infected area of skin. It is a further object of the device to prevent the NO-containing gas from leaking from the delivery device. The method of delivering an effective amount of nitric oxide gas to the infected area of skin kills bacteria and other pathogens and promotes the healing process.

As clearly illustrated, Stenzler never taught, suggested, nor disclosed exposing blood to NO to destroy pathogens.

In 1989 it was discovered that nitric oxide was produced by the endothelium tissue of mammals. It has since been demonstrated that endogenous nitric oxide is a potent modulator for a number of systemic functions in mammals including selective pulmonary vasodilatation, neurotransmission and cytoxic activity over a wide range of microorganisms including bacteria and viruses. Nitric oxide has been known for years as an environmental pollutant and is toxic to mammals at high doses. At minimal concentrations however exogenously supplied (eg. <100 ppm) nitric oxide has selectively been used to treat human patients with a wide range of pulmonary diseases including, but not limited to, chronic bronchitis, asthma, ARDS (Acute Respiratory Disease Syndrome) etc. Nitric oxide has also found utility in its application as both a sterilizing agent and as a bactericidal agent for pathogenic organisms.

Septicemia is a serious, rapidly progressive, life-threatening infection that can arise from infections throughout the body, including infections in the lungs, abdomen, and urinary tract. It may precede or coincide with infections of the bone (osteomyelitis), central nervous system (meningitis), or other tissues. Septicemia can rapidly lead to septic shock and death. Septicemia associated with some organisms such as meningococci can lead to shock, adrenal collapse and disseminated intravascular coagulopathy.

In all examples referenced there is a dosage range of nitric oxide application that needs to be maintained in order to establish efficacy. Accordingly the employment of nitric oxide as a dissolved gas or through selective nitric oxide donors in an extracorporeal circuit allows for the titration of exogenously administered nitric oxide levels required to optimize the therapeutic antimicrobial and bactericidal benefits.

The impact from lost industrial productivity along with its significant impact on the public health sector makes the eradication of biofilms a major goal.

### BRIEF DESCRIPTION OF THE DRAWINGS

FIGS. 1-8 are prior art.

FIG. 9 is a schematic of the present invention.

FIG. 10 is an alternative embodiment of the present invention.

FIG. 11 is photograph of cultured biofilms treated with NO gas and air control as discussed in Example 2.

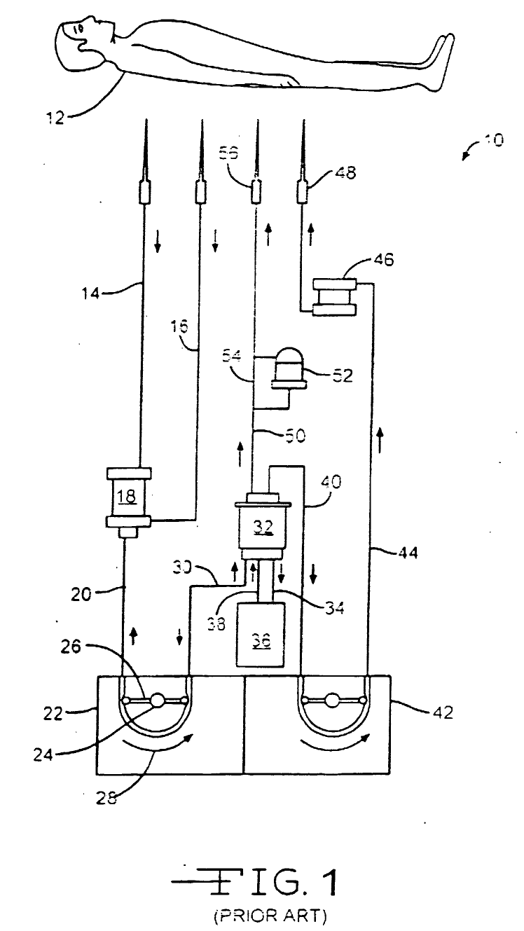

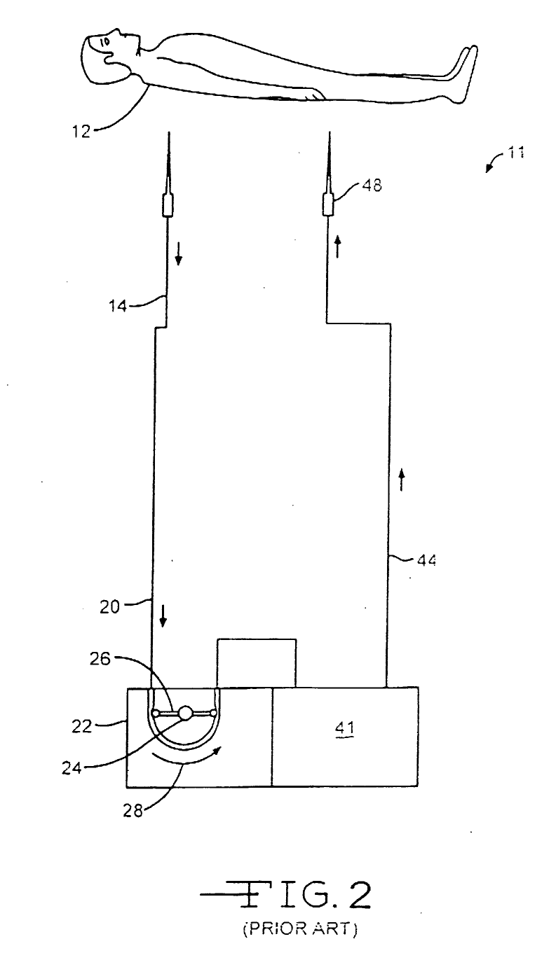

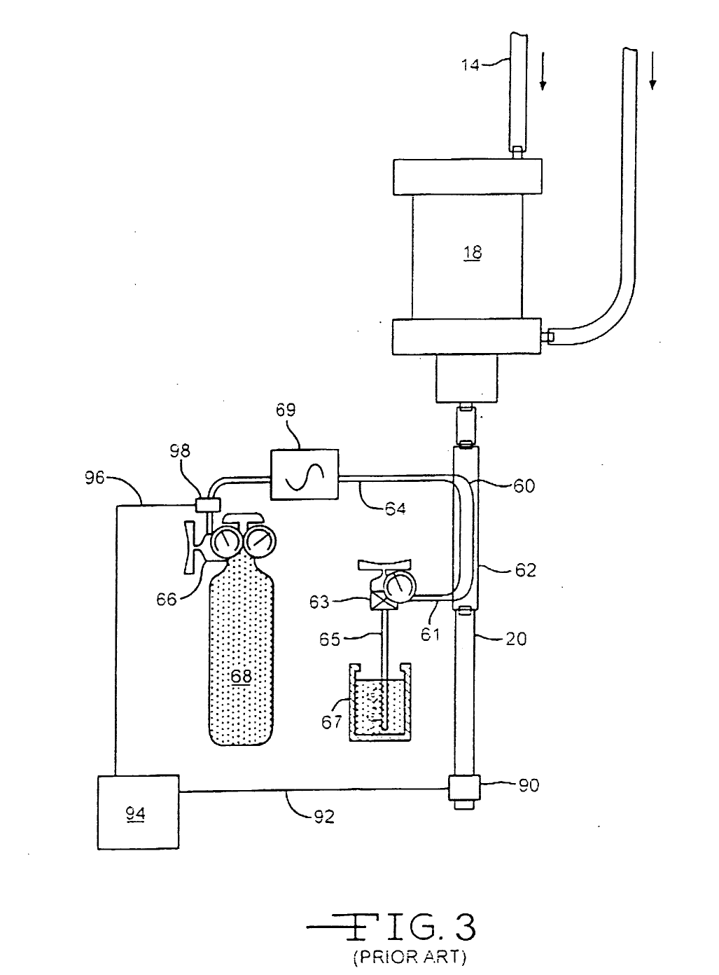

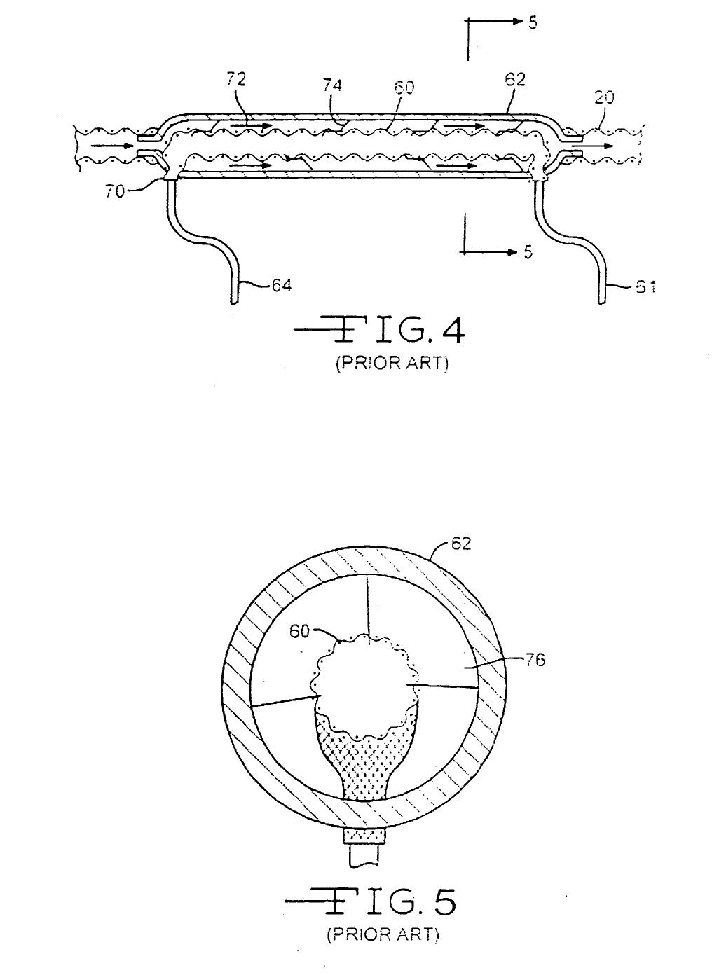

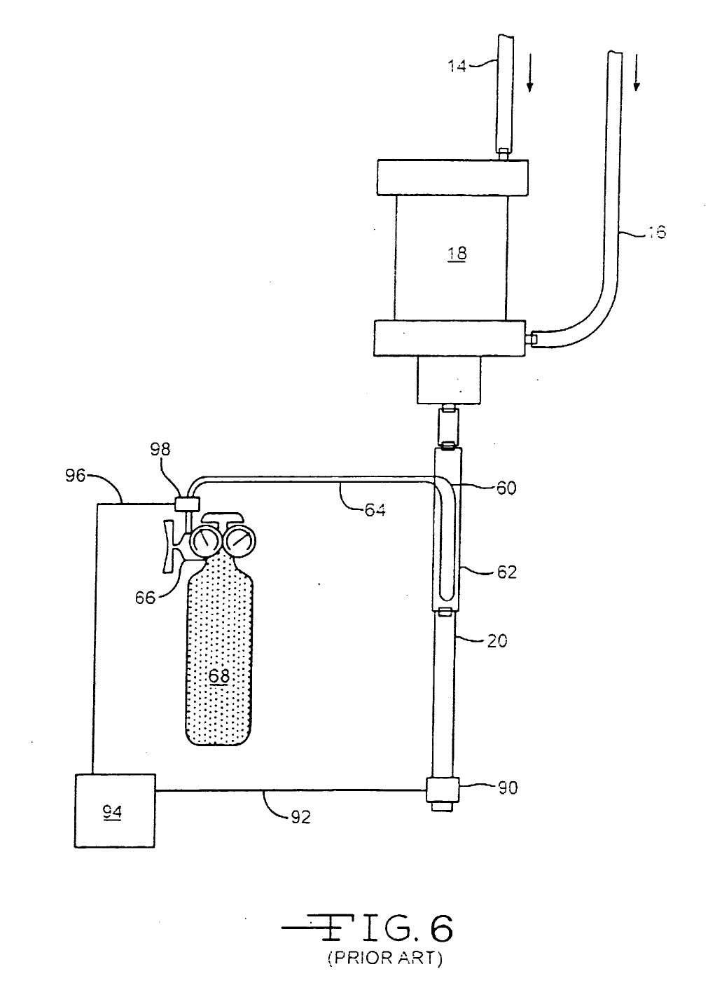

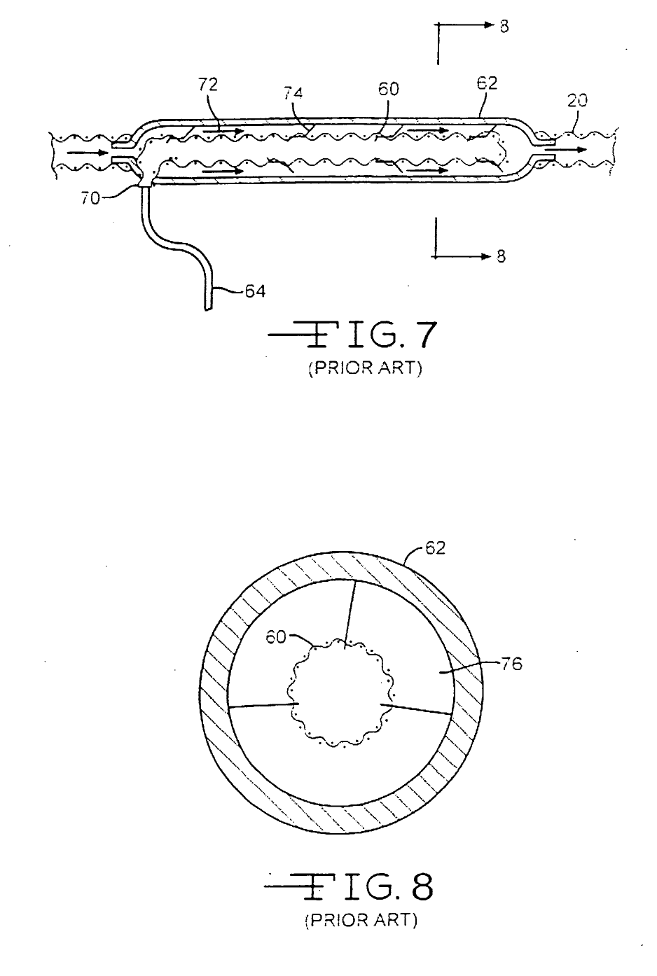

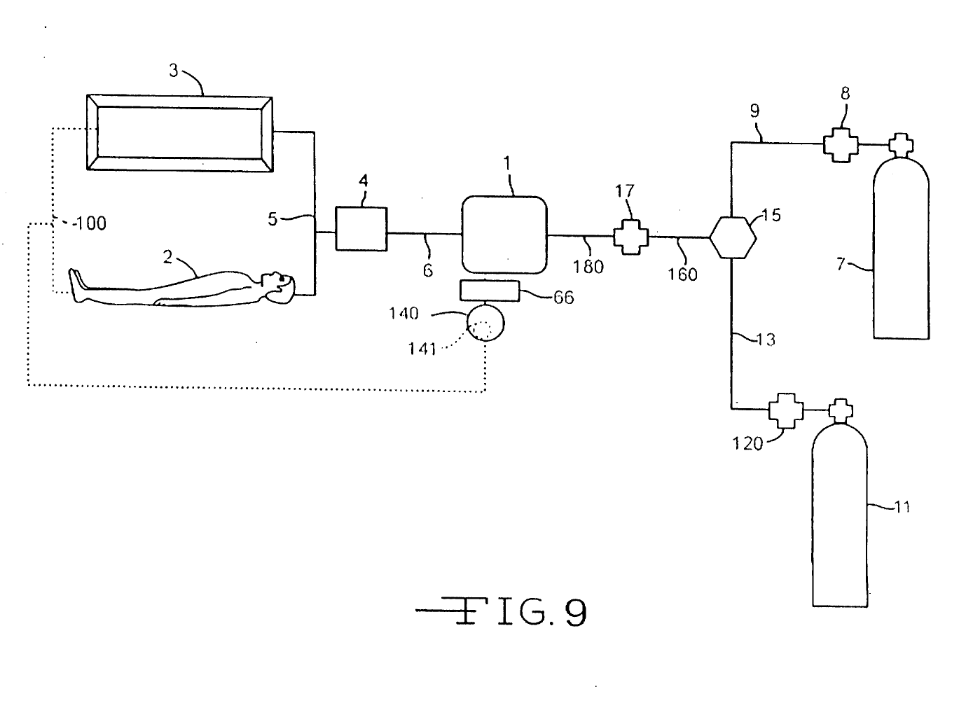

### SUMMARY OF INVENTION

The antimicrobial properties of nitric oxide as a molecule have been well documented. The administration of gaseous nitric oxide as a biocidal moiety is proffered as a de novo treatment in the control and eradication of biofilms. The present invention relates to the use or methods of application of exogenous nitric oxide gas (gNO) as a stand alone biocidal agent or in cohort with any or all adjunct vehicles in the control of biofilms generated by microbial organisms i.e. bacteria, protozoa, amoeba, fungi etc. Further, the present invention introduces the concept of utilization and methods of application of gaseous nitric oxide in control and eradication of biofilm forming microorganisms. Noteworthy areas of application are offered as examples. They include, and are not limited to, air and/or water heating/cooling distribution systems in facilities such as hospitals and laboratories, surfaces of medical devices, household surfaces, dental plaque, dental and/or medical water treatment lines, industrial pipelines, water treatment and distribution facilities and fluids sterilization. Various specialized delivery apparatus will be designed to facilitate nitric oxide gas administration to each specific unique application.

Another embodiment of the invention is a method of treating biofilm comprising: applying a nitric oxide releasing material to a biofilm and allowing gaseous nitric oxide released from the material to contact the biofilm. The nitric oxide releasing material may be placed on the biofilm or may be placed in close proximity to the biofilm. The nitric oxide releasing material and the biofilm may be surrounded by an air impermeable cover. The nitric oxide releasing material may be a polymeric composition or may be a nitric oxide releasing sol-gel coating on a substrate. The concentration of gaseous nitric oxide that contacts the biofilm may be greater than 100 ppm or may be greater than 200 ppm.

Another embodiment of the invention is a method of preventing the formation of a biofilm comprising: applying a nitric oxide releasing material to a surface that may be susceptible to biofilm formation and allowing gaseous nitric oxide released from the material to contact the surface. The nitric oxide releasing material may be placed on the surface or may be placed in close proximity to the surface. The nitric oxide releasing material and the surface may be surrounded by an air impermeable cover. The nitric oxide releasing material may be a polymeric composition or may be a nitric oxide releasing sol-gel coating on a substrate. The concentration of gaseous nitric oxide that contacts the surface may be greater than 100 ppm or may be greater than 200 ppm.

In another embodiment of the invention, nitric oxide releasing compounds, such as NO-donors and upregulators of NO are used to prevent and/or treat a biofilm.

A polymer based paint may be charged with NO molecules applied to a wall in a hospital. The diffusion of the NO molecules from the paint may inhibit or eradicate biofilms. Another embodiment of the present invention is a process of inhibiting or eradicating of biofilms by applying a nitric oxide releasing material to a wall, in a coating, such as in the operating room.

A pharmaceutical agent may be charged with NO molecules, such as by direct exposure to NO gas. The combination of the pharmaceutical agent plus NO may treat, inhibit or eradicate biofilms and other bacteria based conditions or infections.

Another embodiment of the invention is a substantially air-tight container made of substantially gas impermeable material comprising: a nitric oxide releasing material and gaseous nitric oxide at a sufficient concentration to inhibit diffusion of gaseous nitric oxide from the nitric oxide releasing material. The nitric oxide releasing material may be a polymeric composition or may be a nitric oxide releasing sol-gel coating on a substrate. The gas impermeable material may be selected from the group consisting of a metal foil, an aluminized foil laminate, and a laminate, the laminate having at least one materialized layer that includes a material selected from the group consisting of nylon, polypropylene, ethylene vinyl alcohol, polyethylene terephthalate, low density polyethylene, medium density polyethylene and cellophane.

Another embodiment of the invention is a substantially air-tight container made of substantially gas impermeable material comprising: a medical device and a nitric oxide source, wherein nitric oxide molecules from the nitric oxide source contact the medical device within the container and prevent the formation of a biofilm on the medical device. The gas impermeable material may be selected from the group consisting of a metal foil, an aluminized foil laminate, and a laminate, the laminate having at least one materialized layer that includes a material selected from the group consisting of nylon, polypropylene, ethylene vinyl alcohol, polyethylene terephthalate, low density polyethylene, medium density polyethylene and cellophane. The nitric oxide source may be a nitric oxide releasing polymeric composition, a nitric oxide releasing sol-gel coating on a substrate, or gaseous nitric oxide provided at a concentration of greater than about 100 ppm.

The foregoing and additional advantages and characterizing features of the present invention will become clearly apparent upon reading of the ensuing detailed description together with the included experimental model wherein:

### DETAILED DESCRIPTION OF THE INVENTION

The administration of gaseous nitric oxide is viewed as a novel biocidal agent in the efficacious management of numerous biofilm-forming microorganisms with particular emphasis on antibiotic resistant bacteria. The gNO can be administered through a variety of mechanisms. Examples of said administration of gNO are set forth in commonly assigned U.S. patent application Ser. No. 10/658,665, incorporated herein by reference its entirety. In that application, it was reported the examples are as follows:

Referring now to FIG. 9, a gaseous nitric oxide (NO) delivery device 1 is shown connected to a source of infected blood from either a patient 2 or a stored blood source 3, and a pumping system 4, through lines 5, 6.

The nitric oxide (NO) source 7, can be a pressurized cylinder containing nitric oxide (NO) gas, and a nitric oxide flow control valve/pressure regulator 8, delivering nitric oxide (NO) to the gaseous nitric oxide-delivery device 1 through supply tubing 9 and an optional gas blender 15. The infected blood is then exposed to a controlled amount of nitric oxide (NO) by the gaseous nitric oxide (NO) delivery device 1, and the treated blood is then returned to either a patient 2 or a stored blood source 3, through line 100. The treated blood can still carry the nitric oxide when it returns to the patient or the stored blood source. By carrying a sufficient quantity of nitric oxide into the patient, which is completely contrary to the teaching of Igo, the nitric oxide can reduce the pathogens throughout the whole body of the patient.

In FIG. 9, the nitric oxide (NO) gas source 7 is a pressurized cylinder containing nitric oxide (NO) gas. While the use of a pressurized cylinder is the preferable method of storing the nitric oxide (NO) containing gas source 7, other storage and delivery means, such as a dedicated feed line can also be used. Typically the nitric oxide (NO) gas source 7 is a mixture of N₂ and NO. While N₂ is typically used to dilute the concentration of NO within the pressurized cylinder, any inert gas can also be used.

When the NO gas source 7 is stored in a pressurized cylinder, it is preferable that the concentration of NO in the pressurized cylinder fall within the range of about 800 ppm to about 1200 ppm. Commercial nitric oxide manufacturers typically produce nitric oxide mixtures for medical use at around the 1000 ppm range. Extremely high concentrations of NO are undesirable because accidental leakage of NO gas is more hazardous, and high partial pressures of NO tends to cause the spontaneous degradation of NO into nitrogen. Pressurized cylinders containing low concentrations of NO (i.e., less than 100 ppm NO) can also be used in accordance with the device and method disclosed herein. Of course, the lower the concentration of NO used, the more often the pressurized cylinders will need replacement.

FIG. 9 also shows source of diluent gas 11 as part of the NO delivery device 1 that is used to dilute the concentration of nitric oxide (NO) for delivery to the gaseous nitric oxide (NO) delivery device 1 through line 13. The source of diluent gas 11 can contain N2, O₂, air, an inert gas, or a mixture of these gases. It is preferable to use a gas such as N₂ or an inert gas to dilute the NO concentration since these gases will not oxidize the nitric oxide (NO) into NO₂, as would O₂ or air. The source of diluent gas 11 is shown as being stored within a pressurized cylinder. While the use of a pressurized cylinder is shown in FIG. 9 as the means for storing the source of diluent gas 11, other storage and delivery means, such as a dedicated feed line can also be used. The nitric oxide (NO) gas from the nitric oxide (NO) gas source 7 and the diluent gas from the diluent gas source 11 preferably pass through flow control valve/pressure regulators 8,120, to reduce the pressure of gas that is admitted to the gaseous nitric oxide (NO) delivery device 1.

The respective gas streams pass via tubing 9, 13, to an optional gas blender 15. The gas blender 15 mixes the nitric oxide (NO) gas and the diluent gas to produce a nitric oxide (NO)-containing gas that has a reduced concentration of nitric oxide (NO). Preferably, the nitric oxide (NO)-containing gas that is output from the gas blender 15 has a concentration that is less than about 200 ppm. Even more preferably, the concentration of nitric oxide (NO)-containing gas that is output from the gas blender 15 is less than about 100 ppm. The nitric oxide (NO)-containing gas that is output from the gas blender 15 travels via tubing 160 to a flow control valve 17. The flow control valve 17 can include, for example, a proportional control valve that opens (or closes) in a progressively increasing (or decreasing if closing) manner. As another example, the flow control valve 17 can include a mass flow controller. The flow control valve 17 controls the flow rate of the nitric oxide (NO)-containing gas that is input to the gaseous nitric oxide (NO) delivery device 1. The nitric oxide (NO)-containing gas leaves the flow control valve 17 via flexible tubing 180. The flexible tubing 180 attaches to an inlet of the gaseous nitric oxide (NO) delivery device 1. The inlet for 1 might include an optional one-way valve that prevents the backflow of gas.

In one preferred embodiment of the invention, the gaseous nitric oxide (NO) delivery device unit 1 includes an NO sensor 140 that measures the concentration of nitric oxide (NO) in the treated blood or fluid stream. The nitric oxide (NO) sensor 140 and nitric dioxide sensor (15) preferably report the concentrations of NO and NO₂ to a controller within the gaseous nitric oxide (NO) delivery device 1, for source gas flow control and alarm. The sensors, 140, 15, can be chemilluminesence-type, electrochemical cell-type, or spectrophotomentric type sensors.

In a similar embodiment, the present invention takes the nitric oxide gas composition in line 18 and directs the nitric oxide gas composition into a patient's breathing orifice, like a nose and/or mouth. The delivery device can be a conventional gas mask or plastic tubing.

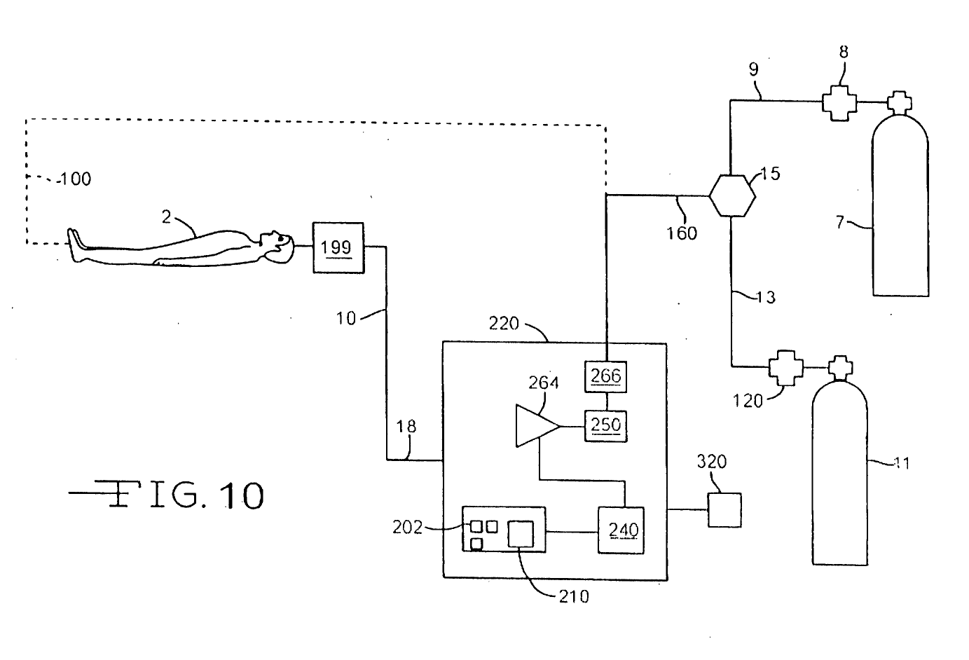

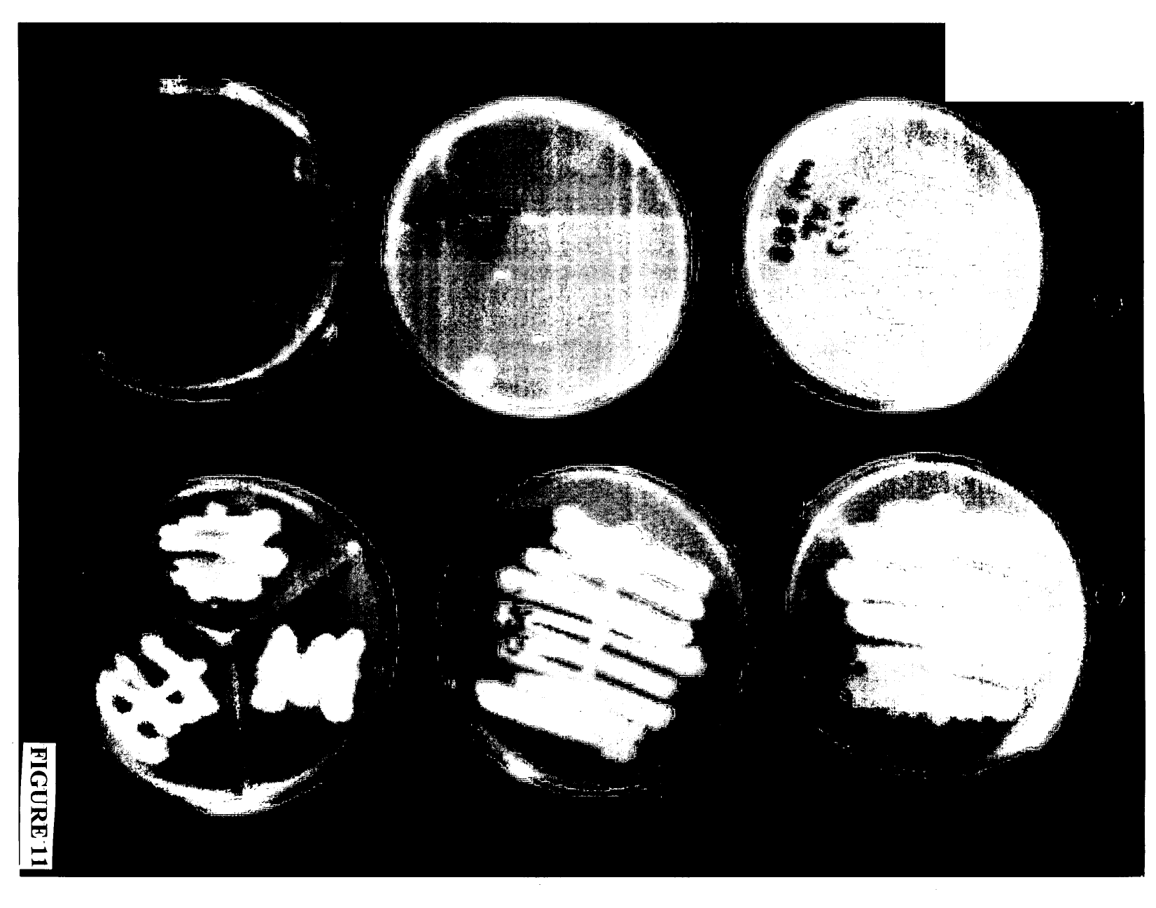

FIG. 10 illustrates a block diagram representation of the device 220, which can be an alternative version of item 17. The device 220 has a power source 220 that provides sufficient voltage and charge to properly operate the device 220. The device 220 also has a main microprocessor 240 that controls the operation of a solenoid valve 260, also within the device 220. The solenoid valve 260 operates in conjunction with operating parameters that are entered via a data entry keypad 201 and the input from a pressure sensor 280.

The operating parameters and the operating status of the device 220 are displayed on an LCD display 210.

The device 220 has a pressure regulator 266. The pressure regulator 266 reduces the pressure of the nitric oxide to less than 100 psi so it can be administered to the patient 2 without damaging the patient's organs, in particular the lungs, from too much pressure.

Calibrating the flow through the solenoid valve 264 is obtained by selecting the pressure of the pressure regulator 266 and controlling the time that the solenoid valve 264 is open. Thereby, the valve 264 allows a precise amount of nitric oxide gas composition to be delivered through the gas delivery line 18, which delivers the nitric oxide to the patient's breathing orifice(s). The pressure sensor 280 is designed to detect a drop in pressure in the gas delivery line 18, when the patient initiates a breath. This pressure drop signals the main processor 240 to open the solenoid valve 264 for a pre-programmed period of time. Among the parameters that are programmed into the device are: Total Breaths. Start Delay, Pulse Time, Pulse Delay, and Retrigger Lock.

The programmable parameters are defined as follows:

Total Breaths: This parameter is the number of breaths programmed into a run of the device 220. Each time a breath is detected as identified above, a pulse of nitric oxide gas composition is injected into the breath of patient 2. Breaths that occur during a locked out time of the predetermined time frame are not counted as breaths. After the programmed number of breaths are counted, the program stops automatically and nitric oxide gas composition is no longer injected into any breaths of the patient. This number can be set anywhere from 0 to unlimited number of breaths. If the number is set at 0 then the auto shutoff is disabled and breaths will be injected with nitric oxide until the user stops the device.

Start Delay: This parameter is the programmed delay time in minutes that the user can set. The injection of nitric oxide gas composition into each breath will begin automatically after “Start Delay” minutes. It will then continue for the number of Total Breaths and then the device 12 stops automatically.

Pulse Time: This parameter is the length of time that the solenoid valve 264 will open for delivery of nitric oxide gas composition. The resolution is 0.1 seconds and the range is 0.1 sec to 0.9 seconds. If the regulator is set at 50 psi then each second of the solenoid valve 264 opening 31 cc of nitric oxide gas composition. If the regulator pressure is set at 30 psi then each 0.1 sec solenoid valve 264 opening represents 21 cc of nitric oxide gas composition. For example, if the regulator is set at 50 psi and the pulse time is set at 0.3 seconds then each detected breath will be injected with a pulse of 0.3 seconds or about 90 cc of nitric oxide gas composition.

Pulse delay: This parameter is the length of time that the machine waits after detecting the beginning of a breath before opening the solenoid valve 264 to inject a pulse of nitric oxide gas composition. This allows the user to control the position of the bolus of nitric oxide gas composition in the breath. For example, if the user sets the solenoid valve 264 at 0.4 seconds, then 0.4 seconds after the beginning of the breath is detected the solenoid valve 264 will open to inject the nitric oxide gas composition pulse.

Retrigger Lock: This parameter is the total time that the machine will ignore new breaths beginning at the detection of a new breath. If this parameter is set at 4.5 seconds then the device 220 will wait, after detecting a breath, for 4.5 seconds before recognizing a news breath. Full or half breaths that are initiated by the patient during this lockout time will not be counted and no nitric oxide gas composition will be injected. If the breath is initiated before the lockout expires and the patient is still inhaling when the lockout expires then it will be recognized as a new breath and it will be counted and injected with nitric oxide gas composition.

The data entry keypad 202 contains five active button switches defined as follows:

START/PULSE KEY: This key is used to start a run. The user is required to confirm the start by pressing an UP key or to cancel by pressing a DOWN key. When a run is in progress, pressing this key will cause the run to pause. The run is then resumed by pressing the UP key or stopping the run by pressing the DOWN key.

UP key: This key is used to confirm the start of the ran, to resume a paused run and also to increment valve charges.

DOWN key: This key is used to cancel a started run, end a paused run and also to decrement valve changes.

NEXT key: This key is used to switch screen pages on the LCD display.

PURGE key: This key is used to open the solenoid valve 264 for two seconds to purge the line. This key is not active during a run. The LCD display can display at least four screen pages, defined as follows:

Each screen page displays a status line. The status variations include NOT RUNNING, WAITING, RUNNING, PAUSED, PURGING and START Pressed.

The main screen page has a row of asterisks on the top line. This is the only screen available when the KEY switch is in the locked position. This screen displays the total breaths detected and also the total breaths that will cause the run to stop.

The second page shows two valves. The first is the START DELAY valve. When the screen first appears the blinking cursor shows the value, which can be changed by pressing either the UP or DOWN key. By pressing the NEXT key switch the cursor to the second value on the screen is TOTAL BREATHS.

The third pace allows the user to change the PULSE DELAY and the PULSE DELAY and the PULSE TIME.

The fourth page allows the user to change the RETRIGGER LOCK.

In any case, this embodiment of the invention allows the nitric oxide gas composition to be injected into a patient's lung, preferably when the patient is inhaling, of a sufficient quantity that nitric oxide is capable of penetrating both the epithelial and capillary basement membranes to allow the nitric oxide to contact the numerous blood cells to reduce pathogens in the blood system and throughout the body.

Other embodiments of the dispenser apparatus of the nitric oxide gas are disclosed in U.S. Pat. No. 6,432,077, which is hereby incorporated by reference herein.

The dispenser can be any device that can apply nitric oxide to any object that can contain a biofilm. The object being selected from a group consisting of a medical device, a conduit for industrial, home, office space, municipal, or medical purposes, and an animal for internal and/or external applications

Alternatively, this latest method can provide the nitric oxide gas continuously, just not when the patient 2 inhales.

In addition the gNO can be directed into application systems via pressurized cylinders to the specific target interface.

Other embodiments of the invention allow for treating a biofilm and preventing the formation of a biofilm using a nitric oxide releasing material. This nitric oxide releasing material can be a nitric oxide releasing polymer or a nitric oxide releasing sol-gel coating on a substrate. As explained above, biofilms associated with various diseases and conditions can form on or within the body of an animal. Biofilms may form on or in the body, such as in the upper respiratory tract, lungs or blood of an animal. Additionally, biofilms can form on the surfaces of medical devices or equipment. The present methods may be used to treat all these types of biofilms.

An example of a nitric oxide releasing sol-gel coating on a substrate is disclosed in the article by Nablo et al., Inhibition of implant-associated infections via nitric oxide release, Biomaterials 26, 6984-6990 (2005), which is hereby incorporated by reference in its entirety as if fully set forth herein. Any coating that releases the NO molecule is also suitable. The NO-releasing coating may be applied to any suitable substrate, such as rubbers, plastics, silica, glass, and polymers.

Any polymeric material can be saturated with nitric oxide gas. For example, a hip joint may be charged with NO molecules in one of the final processing steps of its manufacture. For example, the hip joint could be coated with the NO-releasing coating described above. Thus, once inserted into the body, the NO molecules may linearly diffuse into surrounding tissue over a period of time so as to prevent biofilm formation.

An example of a nitric oxide releasing polymer is disclosed in U.S. Pat. No. 6,432,077, which is hereby incorporated by reference in its entirety as if fully set forth herein. A nitric oxide releasing polymer may be defined by the formula: 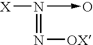
wherein X is an organic or inorganic moiety and X′ is an organic or inorganic substituent, a pharmaceutically acceptable metal center, a pharmaceutically acceptable cation, or the like. The N₂O₂⁻ group is bonded to the polymer through either of both the linking groups of X and X′.

The nitric oxide-releasing N₂O₂⁻ functional group is preferably a nitric oxide/nucleophile adduct, e.g., a complex of nitric oxide and a nucleophile, most preferably a nitric oxide/nucleophile complex which contains the anionic moiety X[N(O)NO]⁻, where X is any suitable nucleophile residue. The nucleophile residue is preferably that of a primary amine (e.g., X=(CH₃)₂CHNH, as in (CH₃)₂CHNH[N(O)NO]Na), a secondary amine (e.g., X=(CH₃CH₂)₂N, as in (CH₃CH₂)₂N[N(O)NO]Na), a polyamine (e.g., X=spermine, as in the zwitterion H₂N(CH₂)₃N⁺(CH₂)₄N[N(O)NO⁻(CH₂)₃NH₂, X=2-(ethylamino)ethylamine, as in the zwitterion CH₃CH₂N[N(O)NO]⁻CH₂CH₂NH₃⁺, or X=3-(n-propylamino)propylamine, as in the zwitterion CH₃CH₂CH₂N[N(O)NO]⁻CH₂CH₂CH₂NH₃⁺), or oxide (i.e., X=O⁻, as in NaO[N(O)NO]Na), or a derivative thereof. Such nitric oxide/nucleophile complexes are capable of delivering nitric oxide in a biologically useable form at a predictable rate.

The nitric oxide releasing material, whether a coating or a polymer, should release nitric oxide gas at a concentration of greater than about 100 ppm gaseous nitric oxide, preferably greater than about 200 ppm gaseous nitric oxide. At a cellular level, this translates into a cellular concentrator of about 200 micro-moles of nitric oxide or greater. Thus, an effective cellular concentrator of the released nitric oxide is about 200 or more micro-moles.

The nitric oxide releasing material is designed to release nitric oxide over a period of time. Diffusion rates may be linear and around 1 ppb to 200 ppm over 7 to 21 days. The concentration of gaseous nitric oxide released from the nitric oxide releasing material may be constant throughout the diffusion from the material.

The nitric oxide releasing materials can be placed directly on a biofilm or surface susceptible to biofilm formation. The nitric oxide releasing materials can also be oriented such that nitric oxide molecules are within close proximity of the biofilm or surface.

Either the nitric oxide releasing sol-gel coating on a substrate or nitric oxide releasing polymer can be formulated into different shapes and sizes to facilitate the release of the NO molecules near the biofilm or targeted surface. For example, they can be formulated into pellets with millimeter to micro-scale diameters or into nano-sized particles. Alternatively, or sheets/planes with dimensions of in the micro or millimeter range are included within the scope of the invention. They can also be formulated into other shapes and sizes depending on how much nitric oxide needs to be released and how they will be used. For example, NO-releasing polymers formulated into 50 mm pellets may be used to treat or prevent biofilms.

Nitric oxide gas and nitric oxide releasing materials may be applied to the blood. As described in U.S. application Ser. Nos. 10/658,665 and 11/445,965, each herein incorporated by reference, treating blood with NO has many benefits such as palette deactivation and the killing of pathogens in the blood. Additionally, the application of nitric oxide gas and nitric oxide molecules from a nitric oxide releasing material, may be used to treat or prevent the formation of biofilms in the blood. The circuitry used to apply gNO is described in application Ser. Nos. 10/658,665 and 11/445,965. The nitric oxide releasing material may be applied to the blood flowing through the circuitry. For example, nitric oxide releasing material may be formulated into the pellets or particles as described above can placed in direct contact with the circulating blood. The pellets or particles may be supplied to the blood individually. Alternatively, the pellets or particles may be supplied in a container or cartridge that releases the pellets or particles such that the pellets or particles contact the blood to treat the blood.

In another embodiment of the invention, a nitric oxide releasing material may be applied to a wall or other surface in the form of a coating. The coating is applied to a wall such as in an operating or hospital room such that the NO molecules may diffuse into the room and prevent or eradiate a biofilm on any surface. The NO molecules act to sterilize the room. This nitric oxide releasing material may be any suitable polymer as described above. This coating may be something that can be wiped off or that is clear such that it can be applied to a wall without interfering with the décor of the room.

In another embodiment of the invention, polymeric based paint may be charged with NO molecules, such that NO can diffuse from the paint on walls and ceilings in hospitals and other facilities to treat biofilms. Any paint made of acrylic resin or vinyl resin, or a combination of both resins, in a liquid form with water as the base may be suitable to charge with NO molecules. The paint may be charged with NO by the direct exposure of the paint to NO gas. The released NO may inhibit or eradicate the formation of a biofilm, or it may be used to treat Staph infections, including MRSA, occur most frequently among persons in hospitals and healthcare facilities (such as nursing homes and dialysis centers).

In another embodiment of the invention, the nitric oxide releasing material may be a pharmaceutical agent (drug) such as an analgesic or anti-inflammatory agent or antimicrobial agent. In this embodiment the drug would be charged with the NO molecule such that upon digestion or injection into the body, NO could diffuse in the body and treat a biofilm or other bacteria. The combination of the drug and the NO molecule is anticipated to give synergistic effects. The drug could be charged with NO by direct exposure of the drug to NO gas.

In another embodiment of the invention, nitric oxide releasing compounds, such as NO-donors and upregulators are used to prevent and/or treat a biofilm. Known nitric oxide-releasing compounds, donors, or upregulators useful in the methods and devices of the invention include, but are not limited to: nitroso or nitrosyl compounds characterized by an—NO moiety that is spontaneously released or otherwise transferred from the compound under physiological conditions(e.g. S-nitroso-N-acetylpenicillamine, S-nitroso-L-cysteine, nitrosoguanidine, S-nitrosothiol); compounds in which NO is a ligand on a transition metal complex, and as such is readily released or transferred from the compound under physiological conditions (e.g. nitroprusside, NO-ferredoxin, NO-heme complex); nitrogen-containing compounds which are metabolized by enzymes endogenous to the respiratory and/or vascular system to produce the NO radical (e.g. arginine, glyceryl trinitrate, isoamyl nitrite, inorganic nitrite, azide and hydroxylamine); anionic diazeniumdiolates (NONOnates); polyethyleneimine (PEI)-based polymers exposed to NO gas; molsidomine; nitrate esters; sodium nitrite; iso-sorbide didinitrate; penta erythritol tetranitrate; nitroimidazoles; complexes of nitric oxide and polyamines; and the NO releasing compounds disclosed in U.S. Pat. No. 5,840,759 and PCT WO 95/09612.

These nitric oxide releasing compounds may be applied to the biofilm in any suitable manner as discussed above, such as directly to a targeted surface or with the use of a substrate to facilitate delivery to the targeted surface.

As another embodiment, the nitric oxide releasing materials can be placed inside of air impermeable containers or packaging with medical devices to prevent the formation of biofilm on the devices. Additionally, the nitric oxide releasing materials would act to sterilize the medical device within the packaging. The packaging or container can be made from a metal foil, an aluminized foil laminate, or a laminate. If made from a laminate, the laminate will have at least one metalized layer that includes a material selected from the group consisting of nylon, polypropylene, ethylene vinyl alcohol, polyethylene terephthalate, low density polyethylene, medium density polyethylene and cellophane. The container can be made to accommodate medical devices of various sizes and shapes. The nitric oxide releasing materials is placed inside the packaging to prevent the formation of the biofilm on the device while the device is sealed within the packaging. Because the medical device has been bathed with nitric oxide molecules within the packaging, it is clean and sterile upon opening the package and using the device.

The nitric oxide molecules within the packaging contact the medical device to prevent the formation of a biofilm on the devices. The source of the nitric oxide molecules can be gaseous nitric oxide, such as a concentration of greater than about 100 ppm gaseous nitric oxide. Furthermore, the source of nitric oxide molecules within the packaging could be from a nitric oxide releasing material. This nitric oxide releasing material can be a nitric oxide releasing polymer or a nitric oxide releasing sol-gel coating on a substrate, as described above. If a nitric oxide releasing material is used, the nitric oxide releasing material would diffuse the NO molecule within the packaging and provide a NO-enriched environment for the device.

Examples of medical devices that could be packaged together with the nitric oxide releasing material include, but are not limited to screws, plates, syringes, surgical tools, dentistry tools, sponges, cardiovascular patches, valves, stents, gloves, prosthetics, sequential compression devices, tissue grafts, artificial blood vessels, electrosurgical accessories, implantable devices, laparoscopes, anthroscopes, sutures, intraocular lenses, vascular grafts, catheters, culdoscopes, hysteroscopes, artificial hearts, pacemakers, pulse generators, fiberoptics, biopsy forceps, endoscopes, endotracheal tubes, and anesthesia breathing circuits.

Another embodiment of this invention is a container for storing nitric oxide releasing materials such that the materials do not release nitric oxide while stored in the container. The container should be sufficiently air-tight and filled with gaseous nitric oxide such that the nitric oxide releasing material within the container is in a state of equilibrium and does not release nitric oxide. The container can be made from the same materials as discussed above.

Thus, in the container comprising the nitric oxide releasing material and the nitric oxide gas, the NO molecules will not diffuse from the nitric oxide releasing material within the container. Thus, the nitric oxide releasing material may be taken out of the packaging and placed upon its targeted surface “fully loaded” with the maximum amount of NO molecules. Gaseous nitric oxide should be provided within the container at a sufficient concentration to inhibit diffusion of gaseous nitric oxide from the nitric oxide releasing material. Such concentration would be greater than about 100 ppm gaseous nitric oxide, preferably greater than about 200 ppm gaseous nitric oxide. Thus, the gaseous nitric oxide is at a pressure above atmospheric pressure within the packaging.

A number of experiments were undertaken to determine the efficacy of various dose concentrations of exogenously applied gaseous nitric oxide on the microorganism Burkholderia cenocepacia. B. cenocepacia is an opportunistic pathogen that plays a role in the formation of biofilms and can cause marked lung infections in cystic fibrosis patients. B. cenocepacia is also associated with increased rates of sepsis and death.

### EXAMPLES

### Example 1

Objective: To determine if exposure to gaseous nitric oxide (gNO) affects the ability of B. cenocepacia C8963 to form a biofilm in a 96-well microtiter dish assay.

Methods: B. cenocepacia C8963, a non-mucoid isolate from a cystic fibrosis (CF) patient, and C9343, a mucoid isolate from the same patient, were spotted on Luria Broth agar and grown at 37° C. overnight. Luria broth containing 0.5% (w/v) casamino acids was dispensed into 96-well polypropylene microtiter dishes (100Φl per well) and the wells inoculated with the C8963 or C9343 using a pin-inoculation device. Blank wells were not inoculated. Dishes were incubated in a humidified, closed plastic container for 24 hours at 37° C. (experiment 1) or in the outer chamber of the matrix incubator (a humidified incubator with controlled air flow) for 27 hours at 37° C. (experiment 2). At 24 hours (experiment 1) or 27 hours (experiment 2), one dish was processed for staining of the bacterial biofilms. The remaining dishes were incubated in the inner treatment arms of the matrix incubator at 37° C. in the presence or absence of 200 ppm gNO. One dish for each of the conditions (+gNO or−gNO) was processed for biofilm staining at 32, 36, and 48 hours.

To stain biofilm growth, planktonic bacterial were removed from the microtitre dishes by discarding media and cells. Biofilms were washed to remove remaining non-adherent bacterial in two successive tap water washes. Water was shaken from the wells and the dishes inverted and tapped vigorously on a stack of paper towels to remove as much water as possible. Adherent growth was stained by adding 125 Φl of a 0.1% (w/v) solution of crystal violet to each well and incubating at room temperature for 15 minutes. Crystal violet was discarded as for the previous washes and excess stain was removed in three successive tap-water washes. Excess water was removed by vigorous tapping as before and the stained dishes allowed to air dry.

To quantitate biofilm formation, 200 Φl of 95% ethanol was added to each well, incubated at room temperature for 15 minutes, and 125 Φl from each well was removed to a clean flat-bottomed polystyrene microtitre dish. The absorbance at 595 nm was read on a Bio-Rad Model 3550 Microplate Reader. The average reading from “Blank” (uninoculated) wells containing only media was subtracted from each “Test” well. The (Test-Blank) averages and standard errors of the mean (SEM) were calculated for each condition and time.

Results: As expected, the mucoid C9343 isolate did not form biofilm under any conditions (Chart 1). This is consistent with its previous behavior since the mucoid exopolysaccharide interferes with adherence to surfaces (1).

Overall, the non-mucoid C8963 isolate continued to form biofilm in the presence of gNO but growth was lower than in the absence of gNO. C8963 biofilm growth in the presence of gNO was greater than in the absence of gNO at 32 hours, but by 36 hours, growth in the presence of gNO was significantly lower than in the absence of gNO in both experiment 1 (Chart 1) and experiment 2 (Chart 2). Biofilm growth remained consistently lower in the presence of gNO for the remaining time points up to 48 hours in both experiments (FIGS. 11 and 12).

In experiment 1, maximum biofilm growth occurred at approximately 36 hours (Chart 1) but in experiment 2, maximum biofilm growth did not occur until 48 hours or more (Chart 2).

Discussion: B. cenocepacia C9343 was a mucoid pulmonary isolate from a CF patient that was previously shorn to the a poor biofilm former (1). B. cenocepacia C8963 was a non-mucoid pulmonary isolate from the same CF patient and was shown to be a competent biofilm former (1). To determine if exposure to gNO affected biofilm formation by these organisms, both were grown in the presence and absence of 200 ppm gNO. The organisms were grown for 24 hours without gNO to establish the biofilm, then exposed to gNO or air only in the final 24 hours of the assay. C9343 served as a negative control since it did not form biofilm under any condition. The presence of gNO did not induce biofilm formation by this organism. For this reason, C9343 was not included in experiment 2.

C8963 formed biofilm in two independent assays. In both cases, introduction of gNO after 24 hours (experiment 1) and 27 hours (experiment 2) resulted in increased biofilm growth at 32 hours compared to biofilm growth in the presence of the carrier gas (air). At first, gNO likely provides a source of nitrogen to the growing bacteria, and that this is advantageous while the effective concentration of gNO dissolved in the media is low. At all subsequent time points, C8963 biofilm growth was lower in the presence of gNO. This implies that once the concentration of gNO equilibrated to 200 ppm within the biofilm systems, it decreased the amount of biofilm formation by C8963 compared to the carrier gas. Thus, gNO acted as a nutrient when present at a low effective concentration and as a biofilm inhibitor at higher effective concentrations.

The maximum amount of C8963 biofilm formation was higher in experiment 2 (A₅₉₅=0.507) than experiment 1 (A₅₉₅=0.441). This difference could be due to different initial inocula received or due to differences in the way the organisms were grown on the first by of the experiment. A more detailed time-course and repetition of the growth conditions from experiment 2 would answer this question.

Gaseous NO affected the biofilm growth of B. cenocepacia C8963 in two ways: at low initial concentrations it enhanced biofilm growth and at the 200 ppm final concentration it inhibited biofilm formation in the 96-well microtiter dish assay.

| | | C8 + | | C9 + | C8 Con | C8 + NO | C9 Con | C9 + NO |
|---|---|---|---|---|---|---|---|---|
| Time (h) | C8 Con | NO | C9 Con | NO | SEM | SEM | SEM | SEM |
| 24 | 0.147 | 0.147 | −0.008 | −0.008 | 0.007 | 0.007 | 0.001 | 0.001 |
| 32 | 0.111 | 0.315 | −0.001 | −0.005 | 0.014 | 0.018 | 0.001 | 0.001 |
| 36 | 0.441 | 0.337 | −0.001 | 0.008 | 0.033 | 0.016 | 0.002 | 0.003 |
| 48 | 0.202 | 0.135 | −0.003 | 0.002 | 0.015 | 0.008 | 0.001 | 0.002 |
| 10 | | | | | | | | |

| Time | C8 | C8 + | C8 Con | C9 + NO |
|---|---|---|---|---|
| (h) | Con | NO | SEM | SEM |
| 27 | 0.076 | 0.076 | 0.008 | 0.008 |
| 32 | 0.257 | 0.282 | 0.014 | 0.015 |
| 36 | 0.383 | 0.252 | 0.020 | 0.025 |
| 48 | 0.507 | 0.334 | 0.023 | 0.027 |

The term “ambient” refers to the gases that surround the targeted interface.

If the nitric oxide is exposed to blood, the nitric oxide can work within the blood for a very brief period of time until it is modified by the hemoglobin. The modification is normally when the nitric oxide attaches to the hemoglobin. Once attached, the nitric oxide is normally not able to destroy a biofilm with the present apparatus.

### Example 2

An experiment was performed to determine the effect of gaseous nitric oxide on biofilms. Biofilms were grown on two tubes over 20-30 days with S. aureus. The then “slimy” tubes were then suspended in saline. Swabs were taken from both tubes and three plates from each tube were made. One set of three plates from one of the tubes was exposed to gaseous nitric oxide at 200 ppm for 24 hours, and the other set of three plates was exposed to air for 24 hours.

After 24 hours the set of plates exposed to air had far more biofilm than the set of plates exposed to gaseous nitric oxide. As shown in FIG. 11, the bottom row of plates was exposed to air while the top row was exposed to nitric oxide. A visual observation of the plates revealed at least a 50% reduction in biofilm in the plates exposed to nitric oxide over the control plates.

It is appreciated that various modifications to the inventive concepts described herein may be apparent to those of ordinary skill in the art without departing from the scope of the present invention as defined by the herein appended claims.

We claim:

1. A method of treating a biofilm comprising:
applying a nitric oxide releasing material to a biofilm; and
allowing gaseous nitric oxide released from the material to contact the biofilm.

2. The method of claim 1, wherein the biofilm was generated by a microbial organism selected from the group consisting of bacteria, protozoa, amoeba, and fungi.

3. The method of claim 1, wherein the biofilm is located on a medical device, medical implant, a conduit for industrial, home, office space, municipal, or medical purposes, or an animal.

4. The method of claim 3, wherein the biofilm is located on or in the body of an animal.

5. The method of claim 1, wherein the biofilm is located on surfaces at facilities selected from the group consisting of hospitals, laboratories, dental and/or medical offices, water treatment facilities, and water distribution facilities.

6. The method of claim 1, wherein the concentration of gaseous nitric oxide that contacts the biofilm is greater than about 100 ppm.

7. The method of claim 6, wherein the concentration of gaseous nitric oxide that contacts the biofilm is greater than about 200 ppm.

8. The method of claim 1, wherein the biofilm is located in the blood of an animal.

9. The method of claim 1, wherein the nitric oxide releasing material is a pharmaceutical agent.

10. The method of claim 1, wherein the nitric oxide releasing material is applied to a wall.

11. The method of claim 1, wherein the nitric oxide releasing material is a polymer.

12. The method of claim 11, wherein the nitric oxide releasing material is a polymer based paint.

13. The method of claim 11, wherein the polymer consists a biopolymeric backbone wherein said backbone is of a protein, and at least one nitric oxide releasing N₂O₂⁻ function group selected from the group consisting of X—N(O)NO] or [N(O)NO—X, wherein X is an organic moiety, covalently bonded to said [N₂O₂], and wherein the [N₂O₂] group is covalently bonded in said polymeric composition through said organic moiety.

14. The method of claim 1, wherein the nitric oxide releasing material is a gaseous nitric oxide releasing sol-gel coating on a substrate.

15. The method of claim 14, wherein the substrate is selected from the group consisting of rubbers, plastics, silica, glass, and polymers.

16. The method of claim 1, wherein the nitric oxide releasing material is placed on the biofilm.

17. The method of claim 1, wherein the nitric oxide releasing material and the biofilm are surrounded by an air impermeable cover.

18. The method of claim 1, wherein the nitric oxide releasing material is selected from NO donors, NO upregulators and combinations thereof.

19. A method of preventing the formation of a biofilm comprising:
applying a nitric oxide releasing material to a surface that may be susceptible to biofilm formation; and
allowing gaseous nitric oxide released from the material to contact the surface.

20. The method of claim 19, wherein the surface is located on a medical device, medical implant, a conduit for industrial, home, office space, municipal, or medical purposes, or an animal.

21. The method of claim 20, wherein the biofilm is located on or in the body of an animal.

22. The method of claim 19, wherein the surface is located at facilities selected from the group consisting of hospitals, laboratories, dental and/or medical offices, water treatment facilities, and water distribution facilities.

23. The method of claim 19, wherein the concentration of gaseous nitric oxide that contacts the biofilm is greater than about 100 ppm.

24. The method of claim 23, wherein the concentration of gaseous nitric oxide that contacts the biofilm is greater than about 200 ppm.

25. The method of claim 19, wherein the nitric oxide releasing material is a pharmaceutical agent.

26. The method of claim 19, wherein the biofilm is located in the blood of an animal.

27. The method of claim 19, wherein the nitric oxide releasing material is applied to a wall.

28. The method of claim 19, wherein the nitric oxide releasing material is a polymer.

29. The method of claim 28, wherein the nitric oxide releasing material is a polymer based paint.

30. The method of claim 28, wherein the polymer consists a biopolymeric backbone wherein said backbone is of a protein, and at least one nitric oxide releasing N₂O₂⁻ function group selected from the group consisting of X—N(O)NO] or [N(O)NO—X, wherein X is an organic moiety, covalently bonded to said [N₂O₂], and wherein the [N₂O₂] group is covalently bonded in said polymeric composition through said organic moiety.

31. The method of claim 19, wherein the nitric oxide releasing material is a gaseous nitric oxide releasing sol-gel coating on a substrate.

32. The method of claim 31, wherein the substrate is selected from the group consisting of rubbers, plastics, silica, glass, and polymers.

33. The method of claim 19, wherein the nitric oxide releasing material is placed on the biofilm.

34. The method of claim 19, wherein the nitric oxide releasing material and the biofilm are surrounded by an air impermeable cover.

35. The method of claim 19, wherein the nitric oxide releasing material is a NO-donor.

36. The method of claim 19, wherein the nitric oxide releasing material is selected from NO donors, NO upregulators and combinations thereof.

37. A substantially air-tight container made of substantially gas impermeable material comprising:
a nitric oxide releasing material; and
gaseous nitric oxide at a sufficient concentration to inhibit diffusion of gaseous nitric oxide from the nitric oxide releasing material.

38. The container of claim 37, wherein the gaseous nitric oxide is at a pressure above atmospheric pressure.

39. The container of claim 37, wherein the gas impermeable material is selected from the group consisting of a metal foil, an aluminized foil laminate, and a laminate, the laminate having at least one metalized layer that includes a material selected from the group consisting of nylon, polypropylene, ethylene vinyl alcohol, polyethylene terephthalate, low density polyethylene, medium density polyethylene and cellophane.

40. The container of claim 37, wherein the nitric oxide releasing material is a polymer.

41. The container of claim 40, wherein the nitric oxide releasing material is a gaseous nitric oxide releasing sol-gel coating on a substrate.

42. The container of claim 41, wherein the substrate is selected from the group consisting of rubbers, plastics, silica, glass, and polymers.

43. The container of claim 37, wherein the concentration of the gaseous nitric oxide is greater than about 100 ppm.

44. A substantially air-tight container made of substantially gas impermeable material comprising:
a medical device; and
a nitric oxide source, wherein nitric oxide molecules from the nitric oxide source contact the medical device within the container and prevent the formation of a biofilm on the medical device.

45. The container of claim 44, wherein the gas impermeable material is selected from the group consisting of a metal foil, an aluminized foil laminate, and a laminate, the laminate having at least one metalized layer that includes a material selected from the group consisting of nylon, polypropylene, ethylene vinyl alcohol, polyethylene terephthalate, low density polyethylene, medium density polyethylene and cellophane.

46. The container of claim 44, wherein the nitric oxide source is polymer.

47. The container of claim 44, wherein the nitric oxide source is a nitric oxide releasing material consisting of a gaseous nitric oxide releasing sol-gel coating on a substrate.

48. The container of claim 47, wherein the substrate is selected from the group consisting of rubbers, plastics, silica, glass, and polymers.

49. The container of claim 44, wherein the medical device is selected from the group consisting of screws, plates, syringes, surgical tools, dentistry tools, sponges, cardiovascular patches, valves, stents, gloves, prosthetics, sequential compression devices, tissue grafts, artificial blood vessels, electrosurgical accessories, implantable devices, laparoscopes, anthroscopes, sutures, intraocular lenses, vascular grafts, catheters, culdoscopes, hysteroscopes, artificial hearts, pacemakers, pulse generators, fiberoptics, biopsy forceps, endoscopes, endotracheal tubes, and anesthesia breathing circuits.

50. The container of claim 44, wherein the nitric oxide source is gaseous nitric oxide in a concentration of greater than about 100 ppm.
